(19) United States
(12) Patent Application Publication
(10) Pub. No.: US 2025/0205521 A1
(43) Pub. Date: Jun. 26, 2025

(54) VACUUM-SEALED ORAL-NASAL MASK

(71) Applicant: Paragon Space Development Corporation, Tucson, AZ (US)

(72) Inventor: Peter Francis Readey, Lebanon, TN (US)

(21) Appl. No.: 18/848,998

(22) PCT Filed: Mar. 23, 2023
(86) PCT No.: PCT/US2023/064877

§ 371 Date: Sep. 20, 2024

Related U.S. Application Data
(60) Provisional application No. 63/324,020, filed on Mar. 25, 2022.

(51) Int. Cl. A62B 18/02; A61G 10/02; A62B 7/02; A62B 18/08

(52) U.S. Cl. CPC A62B 18/025; A62B 7/02; A62B 18/08; A61G 10/026

(57) ABSTRACT

This disclosure provides an oral-nasal face mask with a multi-layer seal surface. The multi-layer seal surface includes one or more porous membranes, one or more open cell foams, or combinations thereof. The multi-layer seal surface engages a user's face so that the oral-nasal face mask encloses a space over the user's nasal openings and mouth. The oral-nasal face mask includes a vacuum line connected to a vacuum source for removal of air, vapor, moisture, and other gases from the multi-layer seal surface, where the vacuum line is fluidly coupled to the one or more open cell foams or the one or more porous membranes. The oral-nasal face mask improves sealing to the user's face while preventing leakage of gas from the oral-nasal face mask and ingress of ambient air.

### INCORPORATION BY REFERENCE

A PCT Request Form is filed concurrently with this specification as part of the present application. Each application that the present application claims benefit of or priority to as identified in the concurrently filed PCT Request Form is incorporated by reference herein in its entirety and for all purposes.

### TECHNICAL FIELD

This disclosure relates to face masks, and more particularly to oral-nasal face masks providing improved contact with a user's face.

### BACKGROUND

Masks for placement over a user's face include nasal face masks, nose and mouth (oral-nasal) face masks, and full face masks, among other types of face masks. Face masks may be worn by various members of the general population including medical personnel, medical patients, laboratory workers, veterinarians, chemical handlers, painters, miners, divers, submariners, and other users. Face masks may serve to prevent the invasive entry of fluids or other particulates from entering the openings of a wearer's mouth, nose, and/or eyes. Face masks may also serve to prevent leakage of air or gas to a surrounding environment. Face masks are increasingly being worn by the general population to limit the spread of infectious diseases such as Covid-19.

In some cases, face masks are used in supplying anesthesia, medication, oxygen, or other gas to a user. The anesthesia, medication, oxygen, or other gas may be supplied to the nose and mouth of the user in an oral-nasal face mask. These face masks may be adapted to conform to the contours of a user's face to prevent leakage as the user breathes in and out. In designing face masks to seal around the user's nose and mouth, it is important to consider factors such as bulk, weight, comfort, and seal effectiveness of the face mask.

### SUMMARY

The systems, methods and devices of this disclosure each have several innovative aspects, no single one of which is solely responsible for the desirable attributes disclosed herein.

One innovative aspect of the subject matter described in this disclosure can be implemented in an oral-nasal face mask. The oral-nasal face mask includes a mask body adapted to engage a user's face, where the mask body comprises a non-porous membrane and a face-contacting sealing membrane, where the face-contacting sealing membrane includes multiple layers of the following: one or more porous membranes, one or more open cell foams, or combinations thereof. The non-porous membrane defines a chamber space over a user's nasal openings and mouth, where the face-contacting sealing membrane is coupled to a vacuum port in the mask body.

In some implementations, the face-contacting sealing membrane comprises the one or more porous membranes and the one or more open cell foams, where at least one porous membrane faces the user's face and at least one open cell foam underlies the at least one porous membrane. In some implementations, the one or more porous membranes and the one or more open cell foams comprise soft materials. In some implementations, the face-contacting sealing membrane conforms to the user's face by a pressure differential generated at an interface between an ambient environment and the face-contacting sealing membrane. In some implementations, the pressure differential prevents leakage of gas from the chamber space to the ambient environment and prevents ingress of ambient air into the chamber space. In some implementations, the mask body is configured to be connected to a low-pressure source via the low-pressure port to generate the pressure differential between the ambient environment and the face-contacting sealing membrane. In some implementations, the low-pressure source comprises a vacuum pump, external reservoirs, outside sources that provide a pressure differential, or Venturi from existing gas flow lines or air supply lines. In some implementations, the face-contacting sealing membrane is configured to facilitate passage of air, vapor, moisture, and other gases, and the face-contacting sealing membrane further comprises collecting channels for capturing the air, vapor, moisture, and other gases for removal through the low-pressure port. In some implementations, the face-contacting sealing membrane is a liner attached and positioned along a peripheral region of the mask body. In some implementations, an inlet port in the mask body is configured to receive inhalation gas into the chamber space and an outlet port in the mask body is configured to discharge exhalation gas from the chamber space. In some implementations, the mask body is configured to be connected to a breathable gas delivery system via the inlet port, and the mask body is configured to be connected to a breathable gas reclamation system via the outlet port. In some implementations, the non-porous membrane includes silicone. In some implementations, the face-contacting sealing membrane has a thickness equal to or less than about 10 μm, and the non-porous membrane has a thickness equal to or greater than about 20 μm.

Another innovative aspect of the subject matter described in this disclosure can be implemented in a face mask. The face mask includes a mask body adapted to engage a user's face, where the mask body comprises a non-porous membrane and a face-contacting seal, where the face-contacting seal comprises at least one porous membrane and at least one open cell foam. The non-porous membrane defines an enclosed cavity over a user's nasal openings and mouth, where the face-contacting seal is coupled to a low-pressure port in the mask body.

In some implementations, each of the at least one porous membrane and the at least one open cell foam comprises soft materials. In some implementations, the mask body is configured to be connected to a low-pressure source via the low-pressure port to generate a pressure differential between an ambient environment and the face-contacting seal. In some implementations, the face-contacting seal comprises multiple layers of open cell foams behind one or more layers of porous membranes.

Another innovative aspect of the subject matter described in this disclosure can be implemented in a method of sealing a face mask to a user's face. The method includes contacting the face mask to the user's face and over a user's nasal openings and mouth, where the face mask includes a mask body comprising a non-porous membrane and a face-contacting seal, where the face-contacting seal includes multiple layers of one or more porous membranes and/or one or more open cell foams. The method further includes generating a pressure differential at an interface between an ambient environment and the face-contacting sealing membrane to create a seal between the user's face and the face mask, where the pressure differential is generated via a low-pressure source coupled to a low-pressure port in the mask body.

In some implementations, the non-porous membrane defines an enclosed cavity over the user's nasal openings and mouth, where the pressure differential prevents leakage of gas from the enclosed cavity to the ambient environment. In some implementations, the one or more porous membranes and the one or more open cell foams comprise soft materials.

### BRIEF DESCRIPTION OF THE DRAWINGS

FIG. 1 shows a schematic illustration of a user wearing a conventional oral-nasal face mask.

FIG. 2 shows a schematic illustration of a user wearing a conventional oral-nasal face mask with a nose wire for improved sealing.

FIG. 3 shows a schematic illustration of divers undergoing decompression cycles in a submarine decompression chamber or pressurized rescue module.

FIG. 4 shows a schematic block diagram of an example multi-person oxygen delivery and reclamation system according to some implementations.

FIG. 5 shows a schematic illustration of multiple divers using example rebreather devices coupled to a multi-person oxygen delivery and reclamation system in a submarine decompression chamber according to some implementations.

FIG. 6A shows a perspective view of an oral-nasal face mask having a face-contacting multi-layer membrane according to some implementations.

FIG. 6B shows a perspective view of the oral-nasal face mask of FIG. 6A with a vacuum port according to some implementations.

FIG. 6C shows the oral-nasal face mask of FIG. 6B illustrating a face-contacting sealing membrane according to some implementations.

FIG. 7 shows a cross-sectional schematic illustration of a face-contacting multi-layer membrane fluidly coupled to a vacuum port in an oral-nasal face mask according to some implementations.

Like reference numbers and designations in the various drawings indicate like elements.

### DETAILED DESCRIPTION

A face mask is worn over the face of a user's head to prevent leakage of gas and prevent entry of contaminants. Full face masks may be fitted over an entire face of a user including the user's eyes, nose, and mouth. Though full face masks can be worn securely with a relatively leak-tight seal, full face masks are often cumbersome and can restrict the user's field of vision. Oral-nasal face masks may be fitted over the nose and mouth of the user that are less cumbersome and less restrictive. However, there are many challenges associated with creating leak-tight seals in oral-nasal face masks.

Conventional oral-nasal face masks, as opposed to full face masks, are generally unable to prevent significant leakage between the mask contour and user's face. This is due in part to variations in individual face geometry, mismatches in mask size/shape, wrinkles, facial hair, improper seating, and slippage. For example, some users may have beards that may present difficulties in creating an effective mask seal. Some users may have unusually contoured shapes that may present difficulties in creating an effective mask seal. Faces with different sizes and geometries make a one-size-fits-all mask infeasible and impractical. Difficulties in creating a leak-tight seal for faces of different sizes and geometries are exacerbated as a user breathes in and out. Various strategies are employed to mitigate leakage between the mask contour and the user's face.

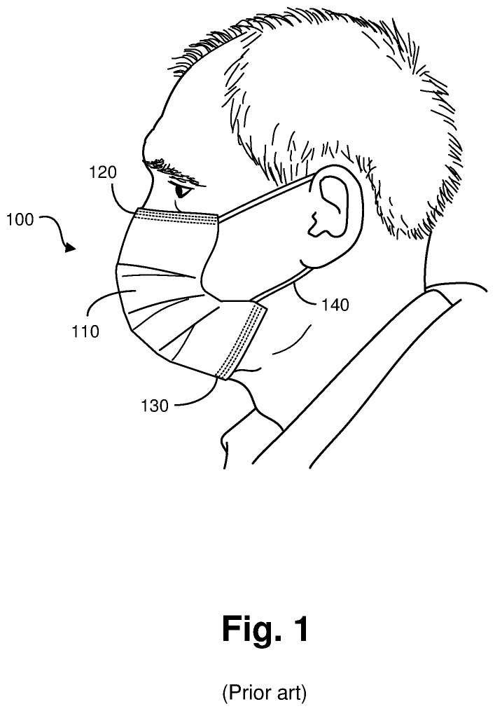

FIG. 1 shows a schematic illustration of a user wearing a conventional oral-nasal face mask. An oral-nasal face mask 100 has a fabric body 110 covering a user's mouth and nose. The oral-nasal face mask 100 has a top edge 120 that may pass along a nasal bridge of a user's face. The oral-nasal face mask 100 also has a bottom edge 130 that may pass under a chin of the user's face. The oral-nasal face mask 100 additionally has straps 140 that wrap around ears of the user's face to secure the oral-nasal face mask 100 over the user's face. The conventional oral-nasal face mask 100 provides inadequate sealing to the user's face.

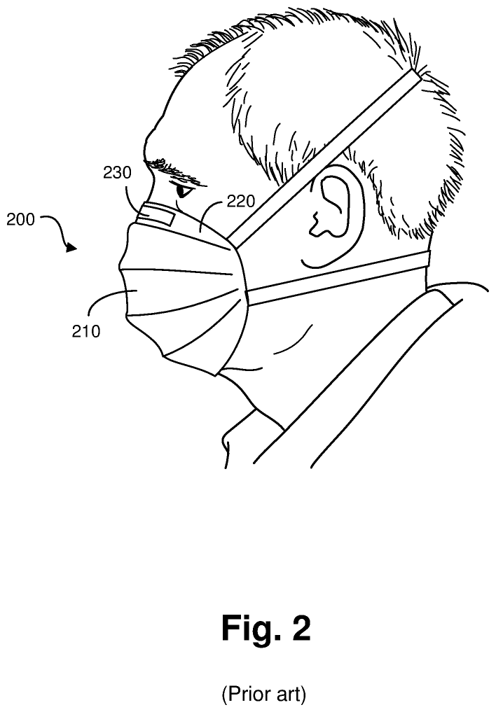

FIG. 2 shows a schematic illustration of a user wearing a conventional oral-nasal face mask with a nose wire for improved sealing. An oral-nasal face mask 200 has a fabric body 210 covering a user's mouth and nose. The oral-nasal face mask 200 has a bendable strip of material 230 positioned at a top edge 220 of the oral-nasal face mask 200. The bendable strip of material 230 may define a nosepiece that bends to fit the contours of the user's nose. The bendable strip of material 230 may serve to properly engage the user's nose to provide a desired anchor and thereby prevent leakage. However, the conventional oral-nasal face mask 200 may still provide inadequate sealing to the user's face that may be caused by variations in individual face geometry, mismatches in mask size/shape, wrinkles, facial hair, improper seating, and slippage.

In some cases, oral-nasal face masks may achieve improved sealing by having straps that tighten to tightly secure the oral-nasal face mask to the user's face. However, strapping tightly is often uncomfortable especially over long periods of time. In some cases, oral-nasal face masks may achieve improved sealing by using materials that more easily adhere and conform to the user's face. This not only accommodates faces of different sizes and geometries, but also accommodates movements of the face. However, such materials ordinarily irritate the user's skin and may even lead to skin rashes, blistering, or ulcerations. Accordingly, improved oral-nasal face masks are needed that have improved sealing and user comfort.

Oral-nasal face masks may be used in a variety of applications. In some applications, oral-nasal face masks enable gas to be provided at positive pressure for consumption by the user. For example, the oral-nasal face masks may serve as breathing masks, where the breathing mask enables oxygen (or other breathable gas) to be supplied to the user. The uses for such breathing masks range from high altitude breathing (i.e., aviation applications) to mining and firefighting applications, to various medical and therapeutic applications.

Oxygen delivery is crucial in a number of life support systems. In medicine, oxygen therapy is used to treat ailments such as emphysema, pneumonia, and some heart disorders. Oxygen can be delivered in a number of ways including a nasal cannula, oral-nasal face mask, full face mask, and a hyperbaric treatment chamber. Hyperbaric treatment chambers are specialized chambers that increase the partial pressure of oxygen around the patient and can treat conditions such as carbon monoxide poisoning and decompression sickness (i.e., the “bends”). With Covid-19 and symptoms of acute respiratory distress syndrome, receiving supplemental oxygen delivery can be life-saving for patients.

Scuba divers and submariners often rely on artificially delivered oxygen. Underwater diving or rebreather systems typically utilize an oxygen supply system for delivery of oxygen or oxygen-enriched gas to a diver, where the flow of oxygen or oxygen-enriched gas can be adjusted. The partial pressure of oxygen (PPO₂) may be controlled as depths vary. When divers or submariners go to depths where they are exposed to elevated atmospheric pressure for long periods of time, they are prone to develop decompression sickness upon returning to normal atmospheric pressures. Miners emerging from a mine may also experience decompression sickness. With decompression sickness, bubbles of inert gas can occur in a person's body as a result of pressure reduction during ascent. To increase the rate of decompression, decompression chambers may supply oxygen or oxygen-enriched gas to divers or submariners to mitigate the risk of developing decompression sickness. As used herein, decompression chambers may also be referred to as recompression chambers.

In addition to mining, diving, and submarining applications, oxygen may be very important to mountain climbers, aviators, and high-altitude parachutists. At reduced atmospheric pressures, air is less dense and less oxygen enters the lungs. The situation can result in a deficiency of oxygen in the blood, or hypoxemia. This can result in heavy breathing, lightheadedness, euphoria, overconfidence, apathy, fatigue, visual disturbances, chest pain, unconsciousness, seizures, and even death. Hypoxemia can be ameliorated by delivery of supplemental oxygen.

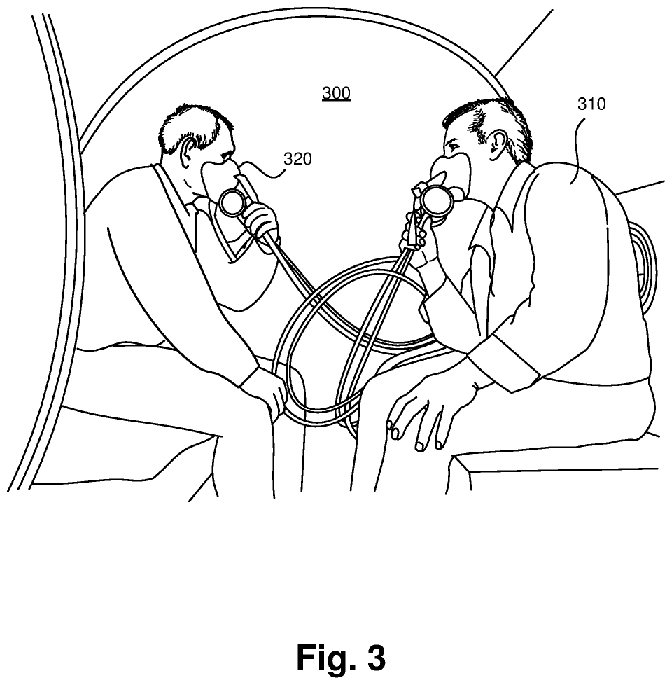

Divers, submariners, aviators, mountain climbers, and others exposed to elevated pressures for long periods of time may need to undergo a procedure called surface decompression. During surface decompression, individuals generally undergo decompression in a chamber. The chamber provides a controlled environment where oxygen can be delivered to each of the individuals at greater partial pressures. FIG. 3 shows a schematic illustration of divers undergoing decompression cycles in a submarine decompression chamber or pressurized rescue module. By way of illustration, trapped underwater submariners or divers 310 may be transferred to a pressurized rescue module (PRM) having a submarine decompression chamber (SDC) 300 prior to returning to the surface. The submarine decompression chamber 300 may hold several personnel, such as 18 to 35 submariners or divers 310 at a time. The submariners or divers 310 in the submarine decompression chamber 300 receive oxygen or oxygen-enriched air according to a certain schedule. The oxygen or oxygen-enriched air may be received via personalized breathing apparatuses or oral-nasal face masks 320. Higher levels of oxygen may be delivered to increase the rate of decompression. In some cases, decompression treatment can take several hours and even days. After decompression treatment, chamber pressure is gradually reduced in the submarine decompression chamber 300. In these applications, it is important for the oral-nasal face masks 320 to be fitted comfortably and securely to each of the submariners or divers 310. This prevents leakage of oxygen so that breathing gas does not contaminate the ambient environment in the submarine decompression chamber 300. This also prevents ambient air from entering the oral-nasal face masks 320 that would contaminate or dilute the breathing gas supplied to the submariners or divers 310.

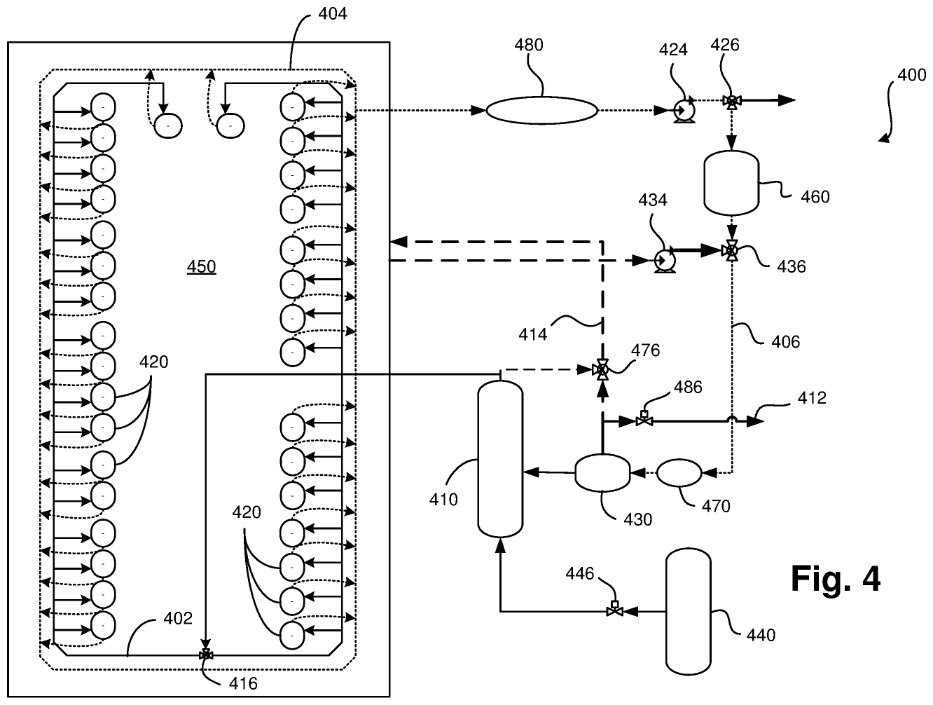

FIG. 4 shows a schematic block diagram of an example multi-person oxygen delivery and reclamation system according to some implementations. Though the present disclosure refers to a multi-person oxygen delivery and reclamation system, it will be understood that the delivery and reclamation system can be applied to any breathable gas and is not limited to only oxygen. As such, the oxygen can be replaced by any breathable gas, and the oxygen delivery and reclamation system can refer to a breathable gas delivery and reclamation system.

The oxygen delivery and reclamation system 400 includes an oxygen-enriched gas supply source 410, a chamber 450, a plurality of rebreather devices 420 adapted for a plurality of users (e.g., divers) in the chamber 450, a gas delivery line 402 fluidly coupled to each of the rebreather devices 420 for receiving inhalation gas, a purge line 404 fluidly coupled to each of the rebreather devices 420 for discharging exhalation air, a recycle line 406 fluidly coupled to the purge line 404, and an oxygen concentrator 430. The oxygen concentrator 430 may be fluidly coupled to the oxygen-enriched gas supply source 410, where the oxygen concentrator 430 provides oxygen-enriched gas to the oxygen-enriched gas supply source 410. In some implementations, an oxygen tank 440 may also be fluidly coupled to the oxygen-enriched gas supply source 410 to provide oxygen (i.e., 100% or high-concentration oxygen) to the oxygen-enriched gas supply source 410. It will be understood that the oxygen from the oxygen tank 440 may not necessarily be 100% oxygen, but may be substantially pure oxygen that is at least 95% oxygen, at least 98% oxygen, or at least 99% oxygen. A gas delivery line 402 may be fluidly coupled to the rebreather device 420 and provide oxygen-concentrated inhalation gas from the oxygen-enriched gas supply source 410 to the rebreather devices 420. Each of the plurality of rebreather devices 420 comprises a face mask (e.g., oral-nasal face mask) configured to be worn by the users. The purge line 404 may be fluidly coupled to the recycle line 406 via one or both of a recycle pump 424 and an electronically actuated valve 426.

The oxygen concentrator 430 may be fluidly coupled to the recycle line 406 for receiving one or both of exhalation air and chamber air. Chamber air may be pulled into the recycle line 406 via one or both of a recycle pump 434 and an electronically actuated valve 436. The oxygen concentrator 430 may convert air (i.e., exhalation air and/or chamber air) received from the recycle line 406 into oxygen-enriched gas and oxygen-depleted gas. The oxygen-enriched gas may be supplied to the oxygen-enriched gas supply source 410. The oxygen-enriched gas supply source 410 may provide desired oxygen levels to each of plurality of users via the gas delivery line 402.

The oxygen delivery and reclamation system 400 may be contained in an enclosed space such as the chamber 450. The chamber 450 may be a vessel such as a pressurized vessel. Examples of pressurized vessels may be submarine decompression chambers, recompression chambers, or pressurized rescue modules. The oxygen-enriched gas supply source 410, the plurality of rebreather devices 420, the purge line 404, the recycle line 406, the oxygen concentrator 430, and the oxygen tank 440 may be located in the chamber 450. However, it will be understood that in certain embodiments one or more of the oxygen-enriched gas supply source 410, the plurality of rebreather devices 420, the purge line 404, the recycle line 406, the oxygen concentrator 430, and the oxygen tank 440 may be located outside the chamber 450.

Oxygen-concentrated inhalation gas is supplied via the gas delivery line 402 from the oxygen-enriched gas supply source 410 to users of the plurality of rebreather devices 420. The oxygen-concentrated inhalation gas is supplied to any of the plurality of rebreather devices 420 as needed. It is not a constant feed. In some embodiments, activation of a valve and controller coupled to the rebreather device 420 may control delivery of the oxygen-concentrated inhalation gas 454 to the user. Depending on a desired oxygen concentration, an oxygen level setpoint may be established for a particular user of the rebreather device 420. The oxygen level setpoint may be user-specified or provided from a control station. A controller may receive an indication of a desired oxygen level to be supplied to a user of a rebreather device 420. The oxygen level setpoint may depend on various factors such as the user's height, weight, age, breathing capacity, medical needs, decompression schedule, etc. In some embodiments, the oxygen level setpoint may be between 21% oxygen by volume and about 100% oxygen by volume. For example, the oxygen-concentrated inhalation gas 454 may be at least 90% oxygen by volume. In some embodiments, the oxygen-concentrated inhalation gas 454 may be a mixture of oxygen and nitrogen or a mixture of oxygen, nitrogen, and water vapor. The oxygen-concentrated inhalation gas 454 may be diluted with chamber air from the chamber 450. This allows the user to be treated with the desired oxygen concentration.

Each rebreather device 420 provides a breathing loop that receives the oxygen-concentrated inhalation gas and releases exhalation gas. In some embodiments, the exhalation gas is replenished of oxygen metabolized by the user within the breathing loop, where the exhalation gas may be scrubbed by a carbon dioxide scrubber in the rebreather device 420 to recirculate breathing gas to the user. In some embodiments, the exhalation gas is recycled of oxygen by purging the exhalation gas out of the breathing loop. The exhalation gas is collected in the purge line 404, where the purge line 404 is connected to the breathing loop or rebreather device 420. The exhalation gas collected in the purge line 404 may be purged in an automated and controlled manner with or without assistance from the user's lungs. The exhalation gas may be either purged to the recycle line 406 or vented out to atmosphere or an environment outside the chamber 450. A purge pump 424 connected to the purge line 404 may be actuated to pull the exhalation gas to the recycle line 406 or to the environment outside the chamber 450. The purge pump 424 may pull a vacuum on the purge line 404 to purge the exhalation gas from the breathing loop. Upon actuation, a pressure differential or vacuum may be generated in the recycle line 406 to pull the exhalation gas from the purge line 404 to the recycle line 406. If recycling of the exhalation gas is not needed, the exhalation gas may be vented to the atmosphere. As a result, the exhalation gas may be circulated for oxygen reclamation or vented even without user effort.

In some implementations, the exhalation air is optionally treated by a hygiene/trace contaminant removal system 480. The hygiene/trace contaminant removal system 480 may serve to clean the exhalation air of contaminants. Prior to oxygen reclamation, the exhalation air may be treated to remove organics, acids, and other contaminant gases. The hygiene/trace contaminant removal system 480 may be downstream of the rebreather devices 420 and upstream of the recycle pump 424.

When purging the exhalation air for recycling, the exhalation air may be optionally collected in a buffer/accumulator tank 460. The buffer/accumulator tank 460 may serve to smooth out a feed of the exhalation air to a carbon dioxide scrubber 470 and/or the oxygen concentrator 430. An electronically or mechanically actuated valve 436 may provide controlled delivery of the exhalation air from the buffer/accumulator tank 460 to the carbon dioxide scrubber 470 or oxygen concentrator 430.

The exhalation gas accumulates in and passes through the recycle line 406 and feeds into the oxygen concentrator 430. In some implementations, a chemical scrubber 470 such as a carbon dioxide scrubber is upstream of the oxygen concentrator 430. The chemical scrubber 470 may remove a chemical such as carbon dioxide from the exhalation gas. In some embodiments, the chemical scrubber 470 may release heat and water vapor as a byproduct of removal of carbon dioxide, thereby heating and humidifying the exhalation gas. The exhalation gas is received by the oxygen concentrator 430, where the oxygen concentrator 430 receives low-purity oxygen and outputs high-purity oxygen. In some implementations, the oxygen concentrator 430 may be an electrochemical oxygen concentrator, a vacuum swing adsorption (VSA) oxygen concentrator, or a pressure swing adsorption (PSA) oxygen concentrator.

In some embodiments, the oxygen concentrator 430 receives a feed of air such as the exhalation gas and outputs oxygen-enriched gas and oxygen-depleted gas. The oxygen concentrator 430 may output the oxygen-enriched gas to the oxygen-enriched gas supply source 410 and output the oxygen-depleted gas to one of the following: (i) the chamber 450 via a chamber line 414 and regulated by an electronically or mechanically actuated valve 476, (ii) the environment outside the chamber 450 via an atmospheric line 412 and regulated by an electronically or mechanically actuated valve 486, or (iii) a storage source (not shown). The oxygen-depleted gas may contain less than about 25% oxygen by volume, less than about 22% oxygen by volume, or between about 17% and about 22% oxygen by volume. The oxygen-depleted gas may contain oxygen balanced by nitrogen. The oxygen-depleted gas may also be referred to as normoxic air. The oxygen concentrator 430 replenishes oxygen in purged exhalation air and/or chamber air so that high-purity oxygen can be recycled to the plurality of users. The oxygen concentrator 430 may concentrate air close to 100% oxygen by volume. For instance, the oxygen concentrator 430 may provide high-purity oxygen at a concentration of at least about 90% oxygen by volume.

Additionally or alternatively, chamber air may be pulled from the chamber 450 into the recycle line 406. Typically, chamber air includes ambient air located in the vessel and surrounding the users of the rebreather devices 420. Chamber air may contain less than about 25% oxygen by volume. Pulling chamber air into the recycle line 406 may serve to regulate ambient pressure in the chamber 450. In some implementations, a controller may receive an indication that an ambient pressure of the chamber 450 is above a threshold value or an indication that an oxygen concentration/partial pressure in the chamber 450 is above a threshold value, and the controller may activate a valve to pull the chamber air from the chamber 450. One or both of a recycle pump 434 and an electronically or mechanically actuated valve 436 may pull the chamber air into the recycle line 406. The chamber air may be pulled into the recycle line 406 by a pressure differential or vacuum. In some implementations, pulling the chamber air into the recycle line 406 may be assisted by a chamber pump 434. The chamber pump 434 may regulate the amount of the chamber air being pulled into the recycle line 406. The electronically or mechanically actuated valve 436 may provide controlled delivery of the chamber air from the chamber 450 to the carbon dioxide scrubber 470 or oxygen concentrator 430. The chamber air may feed from the recycle line 406 into the oxygen concentrator 430. In some embodiments, the chemical scrubber 470 may remove carbon dioxide from the chamber air. The oxygen concentrator 430 receives a feed of the chamber air and outputs oxygen-enriched gas and oxygen-depleted gas. In some implementations, the chamber air is combined with the exhalation gas to form a mixture of the chamber air and the exhalation gas that feeds into the oxygen concentrator 430. In some other implementations, the chamber air and the exhalation gas are fed separately into the oxygen concentrator 430.

In some implementations, an oxygen tank 440 is fluidly coupled to the oxygen-enriched gas supply source 410. The oxygen tank 440 provides oxygen (i.e., 100% oxygen or high-concentration oxygen) to the oxygen-enriched gas supply source 410. In case there is an issue with the recycling of oxygen in the recycle line 406 and/or the oxygen concentrator 430, the oxygen tank 440 may ensure that there is oxygen being fed to the gas delivery line 402. Thus, the oxygen from the oxygen tank 440 may provide oxygen to the oxygen-enriched gas supply source 410. Or, the oxygen from the oxygen tank 440 may supplement the oxygen-enriched gas from the oxygen concentrator 430 to form a more oxygen-concentrated gas mixture in the oxygen-enriched gas supply source 410.

The oxygen delivery and reclamation system 400 provides individualized control of oxygen level setpoints for each of the plurality of rebreather devices 420 adapted for a plurality of users. Different users may require different oxygen concentrations and treatments. Some users may be more sensitive to higher levels of oxygen during treatment or decompression. Some users may require different decompression schedules. Pure oxygen or oxygen-concentrated gas may be delivered from the oxygen-enriched gas supply source 410 to the plurality of rebreather devices 420. Delivery of the pure oxygen or oxygen-concentrated gas may be regulated by the electronically or mechanically actuated valve 416. Oxygen levels for each user can also be influenced by controlling actuation of chamber air intake valves for diluting oxygen with chamber air addition and actuation of purge valves for purging exhalation air from the breathing loop. Thus, a desired oxygen level or specified oxygen level setpoint can be achieved by controlling delivery of high-purity oxygen via the valve 416 and also controlling delivery of chamber air and controlling purging of exhalation air for each breathing loop. This ensures desired oxygen levels or specified oxygen level setpoints are met for each breathing loop.

In the multi-person oxygen delivery and reclamation system 400, each rebreather device 420 is equipped with an intake line or inlet for receiving oxygen-enriched gas from the gas delivery line 402 and is also equipped with an outtake line or outlet for discharging exhalation air to the purge line 404. Each rebreather device 420 facilitates recycling of oxygen without forcing exhalation air to be discharged directly to an ambient environment. The rebreather device 420 may include an oral-nasal face mask fitted over openings of the mouth and nose of each user. Each rebreather device 420 is connected to the oxygen delivery and reclamation system 400 at the gas delivery line 402 for oxygen or oxygen-enriched gas intake and at the purge line 404 for purging exhalation air. In some cases, oxygen can be replaced by any breathable gas so that the rebreather device 420 can be similarly connected to a breathable gas delivery and reclamation system that facilitates recycling of the breathable gas.

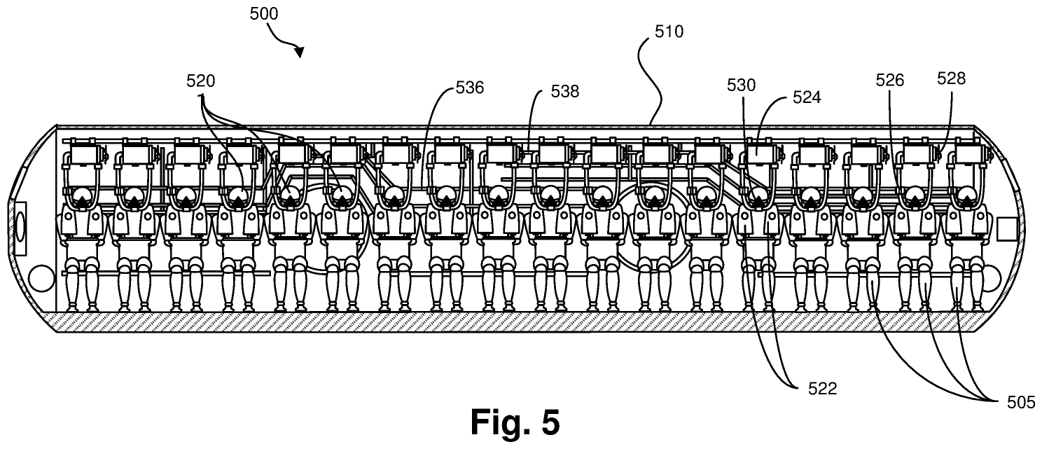

FIG. 5 shows a schematic illustration of multiple divers using example rebreather devices coupled to a multi-person oxygen delivery and reclamation system in a submarine decompression chamber according to some implementations. The submarine decompression chamber 510 is a pressurized vessel providing an enclosed volume for users 505 to undergo decompression cycles. The submarine decompression chamber 510 may also be referred to as a recompression chamber. The submarine decompression chamber 510 may hold a plurality of users 505. The submarine decompression chamber 510 further includes a plurality of rebreather devices 520 adapted for each of the plurality of users 505. Each of the rebreather devices 520 include breathing bags 522 that rest over the shoulders of the users 505. Furthermore, each of the rebreather devices 520 include a mask 530 configured to be worn by the user 505. A carbon dioxide scrubber assembly 524 of each rebreather device 520 is positioned above the users 505 and may be connected to a handrail in the submarine decompression chamber 510. Each of the rebreather devices 520 further includes a purge valve 526 connected to purge tubing 536 of the multi-person oxygen delivery and reclamation system 500. Each of the rebreather devices 520 further includes an oxygen intake valve 528 connected to oxygen tubing 538 of the multi-person oxygen delivery and reclamation system 500.

The present disclosure relates to an oral-nasal face mask that includes a multi-layer seal surface, where the multi-layer seal surface includes a porous membrane and/or open cell foam. The multi-layer seal surface can also be referred to as a face-contacting sealing membrane, where the face-contacting sealing membrane is adapted to form a seal around the user's nasal openings and mouth. A vacuum line or vacuum port is fluidly coupled to the porous membrane and/or open cell foam of the multi-layer seal surface. The vacuum line generates a partial pressure differential between an ambient environment and a vacuum source, so that the multi-layer seal surface seals to a user's face by the partial pressure differential. This creates a “suction” effect so that the oral-nasal face mask conforms to the user's face at the multi-layer seal surface. By keeping the oral-nasal face mask pressed against the user's face at the multi-layer seal surface, breathing gases do not escape from the oral-nasal face mask to the ambient environment and outside gases do not enter into the oral-nasal face mask from the ambient environment. This can be especially important in hyperbaric chambers, submarine decompression chambers, and other applications.

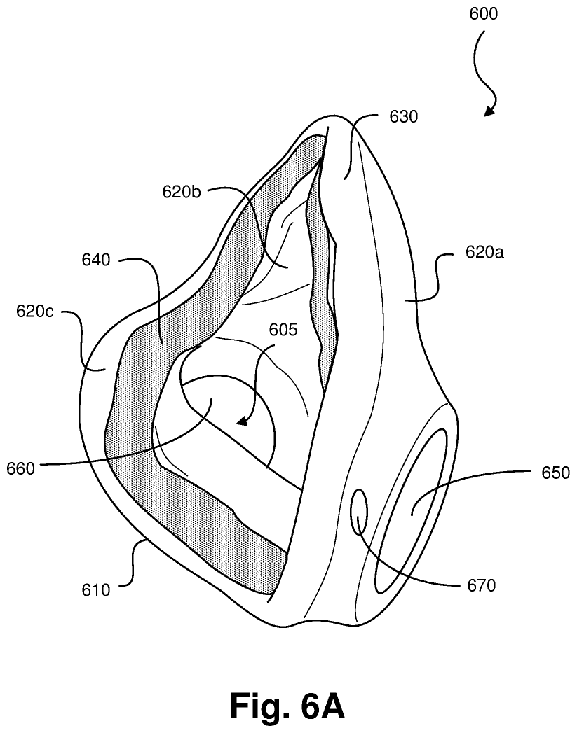

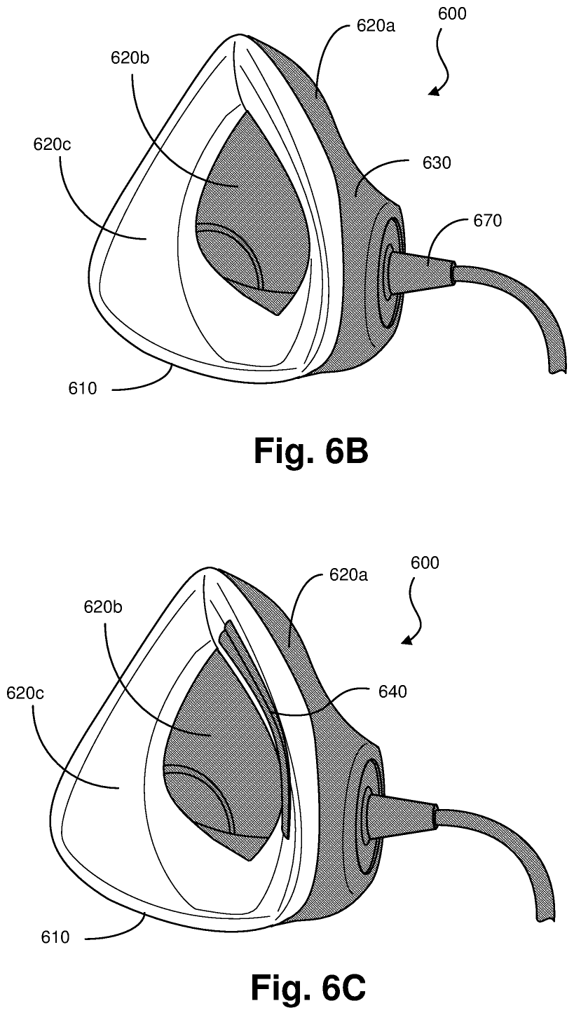

FIG. 6A shows a perspective view of an oral-nasal face mask having a face-contacting multi-layer membrane according to some implementations. FIGS. 6B and 6C provide additional perspective views of the oral-nasal face mask of FIG. 6A. An oral-nasal face mask 600 includes a mask body 610 having an external surface region 620a that defines an outer shell of the oral-nasal face mask 600, an internal surface region 620b that defines an inner chamber space or cavity 605 of oral-nasal face mask 600, and perimeter surface region 620c that defines one or more surfaces that engage a user's face. The mask body 610 has a non-porous membrane 630 and a face-contacting sealing membrane 640. The non-porous membrane 630 surrounds the face-contacting sealing membrane 640. The non-porous membrane 630 is located in the external surface region 620a and the internal surface region 620b of the mask body 610. The non-porous membrane 630 may be provided in portions of the mask body 610 that do not directly interface with the user's face. The face-contacting sealing membrane 640 is located in the perimeter surface region 620c of the mask body 610, where the face-contacting sealing membrane 640 forms a continuous band along perimeter surface region 620c. However, it will be understood that in some other embodiments the face-contacting scaling membrane 640 is non-continuous along the perimeter surface region 620c.

The mask body 610 of the oral-nasal face mask 600 is adapted to cover over nasal openings and a mouth of a user. The mask body 610 may be sized and shaped so that a top portion of the mask body 610 engages the user's nose or nasal bridge, side portions of the mask body 610 engage user's cheeks, and a bottom portion of the mask body 610 engages the user's chin or neck. Thus, the mask body 610 forms a cup over the user's mouth and nasal openings.

A gas inlet passage 650 may define an opening on a first side of the mask body 610 to permit passage of breathing gas into the chamber space 605, and a gas outlet passage 660 may define an opening on a second side of the mask body 610 to permit exhalation gas out of the chamber space 605. The gas inlet passage 650 and the gas outlet passage 660 allows the oral-nasal face mask 600 to be implemented in a breathing loop of a rebreather device. For instance, oxygen-enriched inhalation gas may enter through the gas inlet passage 650 and exhalation gas may exit through the gas outlet passage 660.

A low-pressure port or vacuum port 670 forms an opening in the mask body 610 so that a light vacuum may pull air, vapor, moisture, or other gas by a pressure differential from the face-contacting sealing membrane 640 through the vacuum port 670. The pressure differential may be generated using a low-pressure source (not shown) connected to the vacuum port 670. The low-pressure source may be any space that provides a lower pressure relative to the surrounding environment. The pressure differential is formed between an ambient environment and the low-pressure source. Examples of low-pressure sources include but are not limited to vacuum pumps, external reservoirs, outside sources that provide a pressure differential, and Venturi from existing gas flow lines or air supply lines. The vacuum port 670 is fluidly coupled to the face-contacting sealing membrane 640. The pressure differential at an interface between the ambient environment and the face-contacting sealing membrane 640 provides a continuous seal surface that conforms the face-contacting sealing membrane 640 to the user's face. This improves sealing of the oral-nasal face mask 600 by pressing the face-contacting sealing membrane 640 towards or onto the user's face.

The non-porous membrane 630 may be impermeable to gas or air. The non-porous membrane 630 may include a flexible or deformable plastic material such as silicone. The non-porous membrane 630 can be made of any soft supple and pliable material. The non-porous membrane 630 can form a closed cavity over the user's nasal openings and mouth. The external surface region 620a of the mask body 610 is formed of the non-porous membrane 630 so that air or gas from the ambient environment does not enter into the chamber space 605 of the oral-nasal face mask 600. The internal surface region 620b of the mask body 610 is formed of the non-porous membrane 630 so that breathing gas or exhalation gas does not escape from the chamber space 605 into the ambient environment. The perimeter surface region 620c of the mask body 610 includes the face-contacting sealing membrane 640 and may optionally include portions of the non-porous membrane 630 outlining the face-contacting sealing membrane 640.

The face-contacting sealing membrane 640 serves as a liner along a peripheral region of the mask body 610, where the face-contacting sealing membrane 640 faces towards the user's face. The face-contacting sealing membrane 640 provides an external seal face, which can include soft silicone with perforations over a membrane area or a soft cloth covering molded into a silicone outer seal. In some implementations, the face-contacting sealing membrane 640 has a thickness equal to or greater than about 10 μm. In some implementations, the non-porous membrane 630 has a thickness equal to or greater than about 20 μm. In some instances, the non-porous membrane 630 is thicker than the face-contacting sealing membrane 640.

The face-contacting sealing membrane 640 includes multiple layers of soft materials. The multiple layers of soft materials may include one or more porous layers. The one or more porous layers (e.g., porous membranes, open cell foams) facilitate passage of air, vapor, moisture, and other gases to the vacuum port 670. The face-contacting sealing membrane 640 provides a pressure differential seal where open cell foams and channels reside to collect fluid (i.e., gas or liquid). In some implementations, the multiple layers of soft materials include one or more porous membranes and/or one or more open cell foams. In some instances, open cell foams are made of polyurethane. Open cell foams are generally soft and compressible, and open cell foams are permeable to air, vapor, moisture, and other gases. The layers of porous membranes and open cell foams permit passage of air and other gases. The vacuum port 670 may be fluidly coupled to the layers of porous membranes and open cell foams. The light vacuum or pressure differential pulled by the vacuum port 670 may remove air or other gas passing through the layers of porous membranes and open cell foams. That way, gases entering from the ambient environment may pass through the layers of porous membranes and open cell foams and may be removed to the vacuum port 670. Furthermore, gases escaping from the chamber space 605 of the oral-nasal face mask 600 may be prevented from escaping into the ambient environment by being pulled through the layers of porous membranes and open cell foams to the vacuum port 670. The light vacuum or pressure differential pulled by the vacuum port 670 also creates suction so that the face-contacting sealing membrane 640 presses against the user's face for improved sealing. That way, leakage is prevented between mask contour and the user's face due to individual face geometry, mismatches in mask size/shape, wrinkles, facial hair, improper seating, and slippage during long-term usage. The improved sealing mitigates problems associated with excess perspiration produced over long periods at elevated temperatures. Conventional seals in face masks fail to accommodate for radical geometry changes in a user's face so that leakage in and out is passed directly to the user. However, the oral-nasal mask of the present disclosure, such as the oral-nasal face mask 600, insulates the user from either ingress of air or potential pollution of the ambient environment with unwanted gases (e.g., oxygen)

The oral-nasal face mask 600 may be integrated in a variety of applications such as face masks used in hyperbaric chambers, submarine decompression chambers, aircraft, and hospitals, among other potential applications. In some implementations, the oral-nasal face mask 600 may be integrated in a rebreather device for use in a breathable gas delivery and reclamation system (e.g., oxygen delivery and reclamation system described in FIGS. 4 and 5). The gas inlet passage 650 may receive breathing gas or inhalation gas from a breathable gas delivery system as described above, and the gas outlet passage 660 may discharge exhalation gas to a breathable gas reclamation as described above.

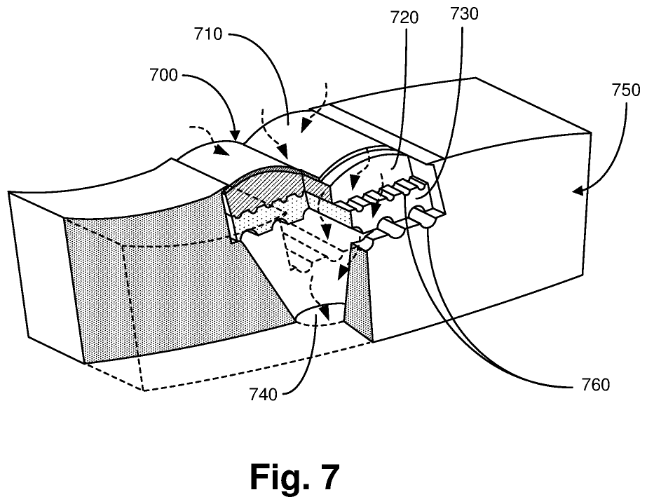

FIG. 7 shows a cross-sectional schematic illustration of a face-contacting multi-layer seal fluidly coupled to a vacuum outlet port in an oral-nasal face mask according to some implementations. The face-contacting multi-layer seal 700 includes a plurality of layers 710, 720, 730 configured to engage a user's face in an oral-nasal face mask. The plurality of layers 710, 720, 730 may be sufficiently permeable or porous to allow passage of air and gases therethrough. Dashed arrows in FIG. 7 symbolize passage of air and gases through the face-contacting multi-layer seal 700. The face-contacting multi-layer seal 700 may be surrounded by a non-porous material 750 of the oral-nasal face mask. A substantial portion of the oral-nasal face mask may be composed of the non-porous material 750. These areas of the oral-nasal face mask are impermeable to air and gases.

The face-contacting multi-layer seal 700 is fluidly coupled to a vacuum port outlet 740. Air, vapor, moisture, and other gases are pulled through the vacuum port outlet 740 by a pressure differential generated between the face-contacting multi-layer seal 700 and the vacuum port outlet 740. The vacuum port outlet 740 is connected to a low-pressure or vacuum line (not shown). Air, vapor, moisture, and other gases pass through the plurality of layers 710, 720, 730 of the face-contacting multi-layer seal 700 and may collect at collecting channels 760. The collected air, vapor, moisture, and other gases are removed through the vacuum port outlet 740 by the pressure differential that pulls the air, vapor, moisture, and other gases through the low-pressure line to a filter or collection chamber installed downstream from the oral-nasal face mask. This effectively draws in otherwise escaping breathing gas so that breathing gas does not escape into the ambient environment and prevents ingress of ambient air from entering into the breathing gas stream.

The face-contacting multi-layer seal 700 includes one or more porous membranes and/or one or more open cell foams. In some embodiments, the face-contacting multi-layer seal 700 may include a porous membrane layer 710. The porous membrane layer 710 may be an outer layer of the face-contacting multi-layer seal 700 that directly contacts or engages with a user's face. The porous membrane layer 710 may include one or more soft materials to provide comfort to the user. Examples of soft materials include elastomers or fluoroelastomers, organic materials, cloth, and the like. The face-contacting multi-layer seal 700 may further include an open cell foam pressure pack 720 below the porous membrane layer 710. The open cell foam pressure pack 720 provides force to seal against the user's skin. In some implementations, the face-contacting multi-layer seal 700 may further include an open cell foam absorbent pack 730 below the open cell foam pressure pack 720. The open cell foam absorbent pack 730 provides a small force to seal against the user's skin and allows fluid to be absorbed into a tighter pore area for improved collection. Accordingly, the open cell foam pressure pack 720 is positioned between the open cell foam absorbent pack 730 and the porous membrane layer 710. A top surface (i.e., facing towards the user's face) of the open cell foam absorbent pack 730 has collection channels 760a that interface with the open cell foam pressure pack 720. A bottom surface (i.e., facing away from the user's face) of the open cell foam absorbent pack 730 has collection channels 760b that interface with the vacuum port outlet 740.

Open cell foams such as open cell foam pressure pack 720 and open cell foam absorbent pack 730 provide light positive pressure while allowing fluid to pass through. Different densities allow more or less positive pressure, thereby providing more or less restrictive flow for fluid. In some embodiments, different densities can control flow of fluid through the face-contacting multi-layer seal 700 and differential pressure. In some implementations, the open cell foam pressure pack 720 may have a different foam density and/or porosity than the open cell foam absorbent pack 730. The foam densities and/or porosity of the porous membrane layer 710, the open cell foam pressure pack 720, and the open cell foam absorbent pack 730 may be optimized to achieve a continuous light contour sealing with the user's face from the pressure differential between the ambient environment and the applied vacuum.

In addition, the face-contacting multi-layer seal 700 provides a better seal by removing moisture from a sealing surface. Often, perspiration from the user causes moisture to build up at the sealing surface that engages the user's face, which can lead to perspiration slippage of the oral-nasal face mask. Perspiration from the user can also cause moisture to escape towards the user's eyes and glasses, causing the user's glasses to fog up. The vacuum or pressure differential generated in the face-contacting multi-layer seal 700 removes moisture from the sealing surface, thereby providing better sealing that can occur from perspiration slippage and thereby preventing moisture from fogging up glasses.

Though the face-contacting multi-layer seal 700 illustrates a porous membrane layer 710, an open cell foam pressure pack 720 underlying/behind the porous membrane layer 710, and an open cell foam absorbent pack 730 underlying/behind the open cell foam pressure pack 720, it will be understood that the face-contacting multi-layer seal 700 can include any combination of porous membranes and open cell foams. Collecting channels 760 may be positioned between layers and/or below layers in the face-contacting multi-layer seal 700. In some implementations, the face-contacting multi-layer seal 700 consists of porous membranes. In some implementations, the face-contacting multi-layer seal 700 consists of open cell foams. In some implementations, the face-contacting multi-layer seal 700 includes a porous membrane sandwiched between open cell foams. In some implementations, the face-contacting multi-layer seal 700 includes an open cell foam sandwiched between porous membranes. In some implementations, the face-contacting multi-layer seal 700 includes an open cell foam with a porous membrane underlying/behind the open cell foam. Other combinations of porous membranes and open cell foams may be used for the face-contacting multi-layer seal 700.

The face-contacting multi-layer seal 700 of FIG. 7 may be incorporated in the oral-nasal face mask 600 of FIGS. 6A-6C. The oral-nasal face mask 600 may be used in a number of applications by users such as medical personnel, medical patients, laboratory workers, chemical handlers, painters, miners, divers, pilots, astronauts, and submariners, among other users. The oral-nasal face mask 600 reduces gas leakage, which in turn supplies more oxygen (or other breathable gas) to the user. This wastes less gas and minimizes oxygen levels being raised in a surrounding chamber or environment. By minimizing leakage, this prevents breathing gas from contaminating the surrounding chamber or environment and limits ambient air from entering into the oral-nasal face mask 600. Otherwise, ingress of ambient air could dilute the oxygen-enriched inhalation gas from an oxygen delivery and reclamation system. The oral-nasal face mask 600 improves sealing to the user's face by providing “suction” and reducing perspiration slippage.

In operation, the oral-nasal face mask 600 may be sealed to a user's face by placing the oral-nasal face mask 600 over the user's nasal openings and mouth, and then creating a pressure differential at an interface between an ambient environment and the face-contacting sealing membrane 640 to create a seal between the user's face and the oral-nasal face mask 600. The oral-nasal face mask 600 is placed so that the face-contacting sealing membrane 640 engages the user's face. The pressure differential can be generated via a low-pressure source coupled to a low-pressure port in the mask body 610 of the oral-nasal face mask 600. The oral-nasal face mask 600 insulates the user from ingress of air or leakage of gas from the oral-nasal face mask 600 to the ambient environment by pulling air or other fluids into internal collection channels.

Although the foregoing disclosed systems, methods, apparatuses, processes, and compositions have been described in detail within the context of specific implementations for the purpose of promoting clarity and understanding, it will be apparent to one of ordinary skill in the art that there are many alternative ways of implementing foregoing implementations which are within the spirit and scope of this disclosure. Accordingly, the implementations described herein are to be viewed as illustrative of the disclosed inventive concepts rather than restrictively, and are not to be used as an impermissible basis for unduly limiting the scope of any claims eventually directed to the subject matter of this disclosure.

What is claimed is:

1. An oral-nasal face mask comprising:
a mask body adapted to engage a user's face, wherein the mask body comprises a non-porous membrane and a face-contacting sealing membrane, wherein the face-contacting sealing membrane includes multiple layers of the following: one or more porous membranes, one or more open cell foams, or combinations thereof, wherein the non-porous membrane defines a chamber space over a user's nasal openings and mouth, wherein the face-contacting sealing membrane is coupled to a vacuum port in the mask body.

2. The oral-nasal face mask of claim 1, wherein the face-contacting sealing membrane comprises the one or more porous membranes and the one or more open cell foams, wherein at least one porous membrane faces the user's face and at least one open cell foam underlies the at least one porous membrane.

3. The oral-nasal face mask of claim 1, wherein the one or more porous membranes and the one or more open cell foams comprise soft materials.

4. The oral-nasal face mask of claim 1, wherein the face-contacting sealing membrane conforms to the user's face by a pressure differential generated at an interface between an ambient environment and the face-contacting sealing membrane.

5. The oral-nasal face mask of claim 4, wherein the pressure differential prevents leakage of gas from the chamber space to the ambient environment and prevents ingress of ambient air into the chamber space.

6. The oral-nasal face mask of claim 4, wherein the mask body is configured to be connected to a low-pressure source via the low-pressure port to generate the pressure differential between the ambient environment and the face-contacting sealing membrane.

7. The oral-nasal face mask of claim 6, wherein the low-pressure source comprises a vacuum pump, external reservoirs, outside sources that provide a pressure differential, or Venturi from existing gas flow lines or air supply lines.

8. The oral-nasal face mask of claim 4, wherein the face-contacting sealing membrane is configured to facilitate passage of air, vapor, moisture, and other gases, and wherein the face-contacting sealing membrane further comprises collecting channels for capturing the air, vapor, moisture, and other gases for removal through the low-pressure port.

9. The oral-nasal face mask of claim 1, wherein the face-contacting sealing membrane is a liner attached and positioned along a peripheral region of the mask body.

10. The oral-nasal face mask of claim 1, wherein an inlet port in the mask body is configured to receive inhalation gas into the chamber space and an outlet port in the mask body is configured to discharge exhalation gas from the chamber space.

11. The oral-nasal face mask of claim 10, wherein the mask body is configured to be connected to a breathable gas delivery system via the inlet port, and wherein the mask body is configured to be connected to a breathable gas reclamation system via the outlet port.

12. The oral-nasal face mask of claim 1, wherein the non-porous membrane includes silicone.

13. The oral-nasal face mask of claim 1, wherein the face-contacting sealing membrane has a thickness equal to or less than about 10 μm, and the non-porous membrane has a thickness equal to or greater than about 20 μm.

14. A face mask comprising:
a mask body adapted to engage a user's face, wherein the mask body comprises a non-porous membrane and a face-contacting seal, wherein the face-contacting seal comprises at least one porous membrane and at least one open cell foam, wherein the non-porous membrane defines an enclosed cavity over a user's nasal openings and mouth, wherein the face-contacting seal is coupled to a low-pressure port in the mask body.

15. The face mask of claim 14, wherein each of the at least one porous membrane and the at least one open cell foam comprises soft materials.

16. The face mask of claim 14, wherein the mask body is configured to be connected to a low-pressure source via the low-pressure port to generate a pressure differential between an ambient environment and the face-contacting seal.

17. The face mask of claim 14, wherein the face-contacting seal comprises multiple layers of open cell foams behind one or more layers of porous membranes.

18. A method of sealing a face mask to a user's face, the method comprising:
contacting the face mask to the user's face and over a user's nasal openings and mouth, wherein the face mask includes a mask body comprising a non-porous membrane and a face-contacting seal, wherein the face-contacting seal includes multiple layers of one or more porous membranes and/or one or more open cell foams; and
generating a pressure differential at an interface between an ambient environment and the face-contacting sealing membrane to create a seal between the user's face and the face mask, wherein the pressure differential is generated via a low-pressure source coupled to a low-pressure port in the mask body.

19. The method of claim 18, wherein the non-porous membrane defines an enclosed cavity over the user's nasal openings and mouth, wherein the pressure differential prevents leakage of gas from the enclosed cavity to the ambient environment.

20. The method of claim 18, wherein the one or more porous membranes and the one or more open cell foams comprise soft materials.
